(19) United States
(12) Patent Application Publication
(10) Pub. No.: US 2023/0398135 A1
(43) Pub. Date: Dec. 14, 2023

(54) COMPOSITIONS INCLUDING CANNABINOID AND USE THEREOF IN THE MANUFACTURE OF A MEDICAMENT FOR THE TREATMENT OF INFLAMMATION IN A SUBJECT

(71) Applicant: ANGROW COMPANY LIMITED, North Kingstown, RI (US)

(72) Inventors: Dengfeng YANG, Shenzhen (CN); Chunfeng CHAI, Shenzhen (CN); Xuan TAN, Birmingham, AL (US)

(73) Assignee: ANGROW COMPANY LIMITED, North Kingstown, RI (US)

(21) Appl. No.: 18/304,373

(22) Filed: Apr. 21, 2023

Related U.S. Application Data
(63) Continuation of application No. PCT/US22/72831, filed on Jun. 8, 2022.

(51) Int. Cl. A61K 31/00; A61P 17/10; A61P 17/02; A61K 9/00

(52) U.S. Cl. CPC A61K 31/658; A61P 17/10; A61P 17/02; A61K 9/0014

(57) ABSTRACT

Compositions including cannabinoid and use thereof are provided in the present disclosure. In some embodiments, the composition may include cannabidiol (CBD), cannabigerol (CBG), and bakuchiol (BAK). A molar concentration ratio of CBD, CBG, and BAK may be 1:1:1 in the composition. A molar concentration of CBD in the composition may be in a range of 1.67 μM to 16.7 μM.

### CROSS-REFERENCE TO RELATED APPLICATIONS

This application is a Continuation of International Application No. PCT/US22/72831, filed on Jun. 8, 2022, the contents of which are hereby incorporated by reference.

### TECHNICAL FIELD

The present disclosure generally relates to compositions comprising cannabinoid, which in some cases can be used to treat inflammation; the present disclosure also relates to compositions comprising cannabinoid and bakuchiol, and in particular, to compositions comprising cannabidiol (CBD), cannabigerol (CBG), and bakuchiol (BAK).

### BACKGROUND

Inflammation is a natural process of the innate immune system, accompanied by an increase in the level of pro-inflammatory mediators and enzymes. Inflammation over a short period is essential for its therapeutic effects on the body. However, prolonged/chronic inflammation can be detrimental as it is related to many chronic conditions and/or diseases such as delayed wound healing, cardiovascular disease, arthritis, autoimmune disorders, and skin problems. For example, repeated acne may be related to prolonged inflammation. It has been shown that cannabinoids can be used to treat inflammation. However, the overall effectiveness and safety of such cannabinoid composition are sometimes in doubt. Thus, it is desirable to provide compositions that include cannabinoids and can treat inflammation effectively and safely.

### SUMMARY

According to an aspect of the present disclosure, a composition is provided. The composition may include cannabidiol (CBD), cannabigerol (CBG), and bakuchiol (BAK). A molar concentration ratio of CBD, CBG, and BAK may be 1:1:1 in the composition. A molar concentration of CBD in the composition may be in a range of 1.67 μM to 16.7 μM.

In some embodiments, a total CBD/CBG/BAK molar concentration of the composition may be in a range of 5 μM to 50 μM. The total CBD/CBG/BAK molar concentration of the composition may be defined by a sum of molar concentrations of CBD, CBG, and BAK in the composition.

In some embodiments, the total CBD/CBG/BAK molar concentration of the composition may be 5 μM, 10 μM, 12.5 μM, 25 μM, or 50 μM.

In some embodiments, the composition may have an inhibitory activity against cyclooxygenase (COX)-2 in vitro.

In some embodiments 2, the composition may have a synergistic anti-inflammatory effect at a target total CBD/CBG/BAK molar concentration in the range of 5 μM to 50 μM. A combination index of CBD, CBG, and BAK in the composition corresponding to the target total CBD/CBG/BAK molar concentration may be less than 1.

In some embodiments, the synergistic anti-inflammatory effect may be measured by inhibitory effect against COX-2 in vitro.

In some embodiments, the target total CBD/CBG/BAK molar concentration of the composition may be 10 μM.

In some embodiments, the composition may be configured to suppress activity of a pro-inflammatory cytokine.

In some embodiments, the pro-inflammatory cytokine may include at least one of interleukin 1β (IL-1β) or tumor necrosis factor alpha (TNF-α).

In some embodiments, the composition may have an antioxidant activity.

In some embodiments, the composition may further include an active component for treating acne or repairing acne scars.

In some embodiments, the active component may include at least one of a hydroxy acid, an additional cannabinoid, or a cannaflavin.

In some embodiments, the additional cannabinoid may include at least one of cannabidiolic acid (CBDA) or cannabigerolic acid (CBGA). The cannaflavin may include at least one of cannaflavin A or cannaflavin B.

According to another aspect of the present disclosure, a method of treating inflammation in a subject is provided. The method may include administering a composition to the subject suffering from a disease related to inflammation. The composition may include cannabidiol (CBD), cannabigerol (CBG), and bakuchiol (BAK). A molar concentration ratio of CBD, CBG, and BAK may be 1:1:1 in the composition. A molar concentration of CBD in the composition may be in a range of 1.67 μM to 16.7 μM.

In some embodiments, a total CBD/CBG/BAK molar concentration of the composition may be in a range of 5 μM to 50 μM. The total CBD/CBG/BAK molar concentration of the composition may be defined by a sum of molar concentrations of CBD, CBG, and BAK in the composition.

In some embodiments, the composition may have a synergistic anti-inflammatory effect at a target total CBD/CBG/BAK molar concentration in the range of 5 μM to 50 μM. A combination index of CBD, CBG, and BAK in the composition corresponding to the target total CBD/CBG/BAK molar concentration may be less than 1.

In some embodiments, the disease of the subject may be related to chronic inflammation.

In some embodiments, the disease of the subject may include repeated acne.

In some embodiments, the composition may be administered to the subject via at least one of an oral administration, an injection administration, or a topical administration.

According to another aspect of the present disclosure, use of a composition in the manufacture of a medicament for the treatment of inflammation in a subject is provided. The use may be characterized in that the composition comprises cannabidiol (CBD), cannabigerol (CBG), and bakuchiol (BAK). A molar concentration ratio of CBD, CBG, and BAK may be 1:1:1 in the composition. A molar concentration of CBD in the composition may be in a range of 1.67 μM to 16.7 μM.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities, and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. It should be noted that the drawings are not to scale. These embodiments are non-limiting exemplary embodiments, in which like reference numerals represent similar structures throughout the several views of the drawings, and wherein:

FIG. 1 is a bar diagram illustrating COX-2 inhibitory effects of CBD and CBG used individually at different molar concentrations according to some embodiments of the present disclosure;

FIG. 2 is a bar diagram illustrating COX-2 inhibitory effects of CBD and CBG used individually and in combination at different molar concentrations according to some embodiments of the present disclosure;

FIGS. 3A and 3B are curve diagrams illustrating COX-2 inhibitory effects of CBD and CBG used individually and in combination at different molar concentrations corresponding to FIG. 2 according to some embodiments of the present disclosure;

FIG. 4 is a bar diagram illustrating COX-2 inhibitory effects of some personal care ingredients used individually at 50 μM according to some embodiments of the present disclosure;

FIGS. 5 and 6 are bar diagrams illustrating COX-2 inhibitory effects of CBD, CBG, and BAK used individually and in combination at different molar concentrations according to some embodiments of the present disclosure;

FIGS. 7 and 8 are bar diagrams illustrating exemplary abilities of CBG, BAK, and a composition of CBD, CBG, and BAK at a molar concentration of 1:1:1 to suppress activities of pro-inflammatory cytokines according to some embodiments of the present disclosure;

FIG. 9 is a bar diagram illustrating exemplary abilities of a composition of CBD, CBG, and BAK at a molar concentration of 1:1:1 at different molar concentrations to suppress activity of IL-113 according to some embodiments of the present disclosure;

FIG. 10 is a bar diagram illustrating antioxidant effects of CBD, CBG, and BAK used individually according to some embodiments of the present disclosure;

FIG. 11 is a diagram illustrating an exemplary target network of CBD, CBG, and BAK according to some embodiments of the present disclosure; and

FIG. 12 is a diagram illustrating an exemplary protein-protein interaction (PPI) network according to some embodiments of the present disclosure.

### DETAILED DESCRIPTION

The following description is presented to enable any person skilled in the art to make and use the present disclosure and is provided in the context of a particular application and its requirements. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but is to be accorded the widest scope consistent with the claims.

The terminology used herein is to describe particular example embodiments only and is not intended to be limiting. As used herein, the singular forms “a,” “an,” and “the” may be intended to include the plural forms as well, unless the context clearly indicates otherwise. It will be further understood that the terms “comprises,” “comprising,” “includes,” “including,” “consist of,” and/or “consist essentially of” when used in this specification, specify the presence of stated features, integers, steps, operations, elements, compositions, and/or components, but do not preclude the presence or addition of one or more other features, integers, steps, operations, elements, compositions, components, and/or groups thereof. For example, a composition of the present disclosure may comprise, consist essentially of, or consist of, essential components as well as optional or additional ingredients described herein, but only if the optional or additional ingredients do not materially alter the basic and novel characteristics of the claimed compositions or methods.

As used herein, the term “ratio” may refer to a molar concentration ratio, unless specifically stated otherwise. The number of significant digits conveys neither a limitation on the indicated amounts nor the accuracy of the measurements. All numerical amounts should be understood to be modified by the word “about” unless otherwise specifically indicated. As used herein, the term “about” and its grammatical equivalents in relation to a reference numerical value and its grammatical equivalents as used herein can include a range of values plus or minus 10% from that value, such as a range of values plus or minus 10%, 9%, 8%, 7%, 6%, 5%, 4%, 3%, 2%, or 1% from that value. For example, the amount of 10 may include amounts from 9 to 11. All numeric ranges are inclusive of narrower ranges and combinable; delineated upper and lower range limits are interchangeable to create further ranges not explicitly delineated.

The term “active component” may refer to a compound that, when applied to a target object, provide a benefit or improvement to the target object. The target object herein may include a user or a portion thereof (e.g., keratinous tissue of the user, a target region of the keratinous tissue, tooth, or the like, or any combination thereof. As used herein, the term “keratinous tissue” may refer to keratin-containing layers disposed as the outermost protective covering of mammals which includes, but is not limited to, skin, hair, nails, cuticles, etc. Accordingly, the active component herein may be used for skincare, haircare, fingernail care, oral care, the like, or any combination thereof.

The term “apply” or “application,” as used in reference to a composition, may refer to applying or spreading the composition onto a skin surface (such as the epidemics), the hair, a surface of a fingernail, the surface of the tooth, etc. of the user.

The term “derivative” may refer to a molecule similar to that of another one, but differing from it with respect to a certain functional moiety (e.g., esters, ethers, amides, amines, carboxylic acids, hydroxyls, acetyls, thiols, halogens, thiols, and/or salt derivatives of the relevant molecule).

The term “dermatologically acceptable” may refer to that the compositions or components thereof are suitable for use in contact with mammalian keratinous tissue without undue toxicity, incompatibility, instability, allergic response, and the like.

The term “effective amount” may refer to an amount of a compound or composition sufficient to significantly induce a positive benefit to keratinous tissue, such as health, appearance, and/or feel benefit, including, independently or in combination, the benefits disclosed herein, but low enough to avoid serious side effects (i.e., to provide a reasonable benefit to risk ratio, within the scope of sound judgment of the skilled artisan). An effective amount of a cannabinoid may be an amount sufficient to regulate a desired condition of mammalian keratinous tissue when topically applied thereto in a composition (e.g., a personal care composition) over the course of a treatment period.

The term “personal care composition” may refer to a topical composition for regulating a condition of mammalian keratinous tissue (e.g., skin, hair, fingernails) and/or tooth. Exemplary personal care compositions may include skin creams, lotions, and serums; shave prep compositions; body washes; deodorants and antiperspirants, shampoos; conditioners; toothpaste, mouthwashes; or the like, or any combination thereof. As used herein, the term “regulating the condition of mammalian keratinous tissue and/or tooth” may refer to improving the appearance and/or feel of keratinous tissue and/or tooth.

The term “topical” may refer to a composition that is intended to be applied to a bodily surface such as skin, hair, and fingernail.

The term “composition(s) containing cannabinoids and bakuchiol (BAK) (e.g., personal care products)” may refer to any compositions that contain the cannabinoids and BAK. Preferred compositions may include compositions used for regulating the condition of the skin, even more preferably reducing the appearance of skin aging, reducing the appearance or occurrence of skin acne, treating skin acne, and/or repairing acne scars. The compositions containing cannabinoids and BAK herein may also exhibit an absence of significant (e.g., consumer-unacceptable) skin irritation (also referred to as being safe) and good aesthetics.

The term “signs of skin aging” may include, but are not limited to, all outward visibly and tactilely perceptible manifestations as well as any other macro or micro effects due to skin aging. Such signs may be induced or caused by intrinsic factors or extrinsic factors, e.g., chronological aging and/or environmental damage. These signs may result from processes that include, but are not limited to, the development of textural discontinuities such as wrinkles and coarse deep wrinkles, skin lines, crevices, bumps, large pores (e.g., associated with adnexal structures such as sweat gland ducts, sebaceous glands, or hair follicles), or unevenness or roughness, loss of skin elasticity (loss and/or inactivation of functional skin elastin), sagging (including puffiness in the eye area and jowls), loss of skin firmness, loss of skin tightness, loss of skin recoil from deformation, discoloration (including undereye circles), blotching, sallowness, hyperpigmented skin regions such as age spots and freckles, keratoses, abnormal differentiation, hyper keratinization, elastosis, collagen breakdown, and other histological changes in the stratum corneum, dermis, epidermis, the skin vascular system (e.g., telangiectasia or spider vessels), and underlying tissues, especially those proximate to the skin.

These and other features, and characteristics of the present disclosure, as well as the methods of operation and functions of the related elements of structure and the combination of parts and economies of manufacture, may become more apparent upon consideration of the following description with reference to the accompanying drawing(s), all of which form a part of this specification. It is to be expressly understood, however, that the drawing(s) are for the purpose of illustration and description only and are not intended to limit the scope of the present disclosure. It is understood that the drawings are not to scale.

According to an aspect of the present disclosure, a composition (also referred to as “MIX” or “Mix”) (e.g., a personal composition) containing one or more cannabinoids and BAK is provided. As used herein, the term “cannabinoid” may refer to a chemical compound belonging to a class of secondary compounds commonly found in plants of genus cannabis, but also encompasses synthetic and semisynthetic cannabinoids. In some embodiments, a cannabinoid described herein may refer to a “purified” cannabinoid which is greater than about a preset pure (e.g., 70% pure, 75% pure, 80% pure, 85% pure, 90% pure, 91% pure, 92% pure, 93% pure, 94% pure, 95% pure, 96% pure, 97% pure, 98% pure, 99% pure, etc). For example, the term “purified” may refer to a cannabinoid that is separated from the plant matter from which it was derived.

In some embodiments, the one or more cannabinoids may include cannabidiol (CBD), cannabigerol (CBG), cannabichromene(CBC), cannabinol (CBN), tetrahydrocannabinol (THC), cannabidivarin (CBDV), tetrahydrocannabivarin (THCV), or the like, or any combination thereof. For example, the one or more cannabinoids may include at least two cannabinoids such as CBD and CBG. That is, the composition may include CBD, CBG, BAK, etc. It should be noted that the composition may include additional cannabinoids other than the CBD and CBG, which is for illustration purposes and not intended to limit the scope of the present disclosure.

In some embodiments, a molar concentration ratio of CBD, CBG and BAK may be (0.5-5):(0.5-5):(0.5-5) in the composition. Merely by way of example, the molar concentration ratio of CBD, CBG, and BAK may be 1:1:1 in the composition. That is, a molar concentration of CBD in the composition, a molar concentration of CBG in the composition, and a molar concentration of BAK may be equal to or substantially equal to each other. In some embodiments, the molar concentration of CBD, CBG, or BAK in the composition may be greater than 0.5 μM. For example, the molar concentration of CBD, CBG, or BAK in the composition may be in a range of 1.67 μM to 16.7 μM. For brevity, a sum of molar concentrations of CBD, CBG, and BAK in the composition may be defined as a total CBD/CBG/BAK molar concentration of the composition. Accordingly, when the molar concentration of CBD, CBG, or BAK in the composition is in the range of 1.67 μM to 16.7 μM, the molar concentration of the composition may be in a range of 5 μM to 50 μM. For example, the molar concentrtion of the composition may be 5 μM, 7.5 μM, 10 μM, 12.5 μM, 15 μM, 25 μM, 30 μM, 40 μM, 45 μM, 50 μM, etc. In some embodiments, the molar concentration of the composition may be greater than 50 μM, which is not limited herein. In some embodiments, the composition with the total CBD/CBG/BAK molar concentration of a specific value may also be referred to as the composition at the specific value. For example, the composition of the total CBD/CBG/BAK molar concentration of 50 μM may also be referred to as the composition at 50 μM.

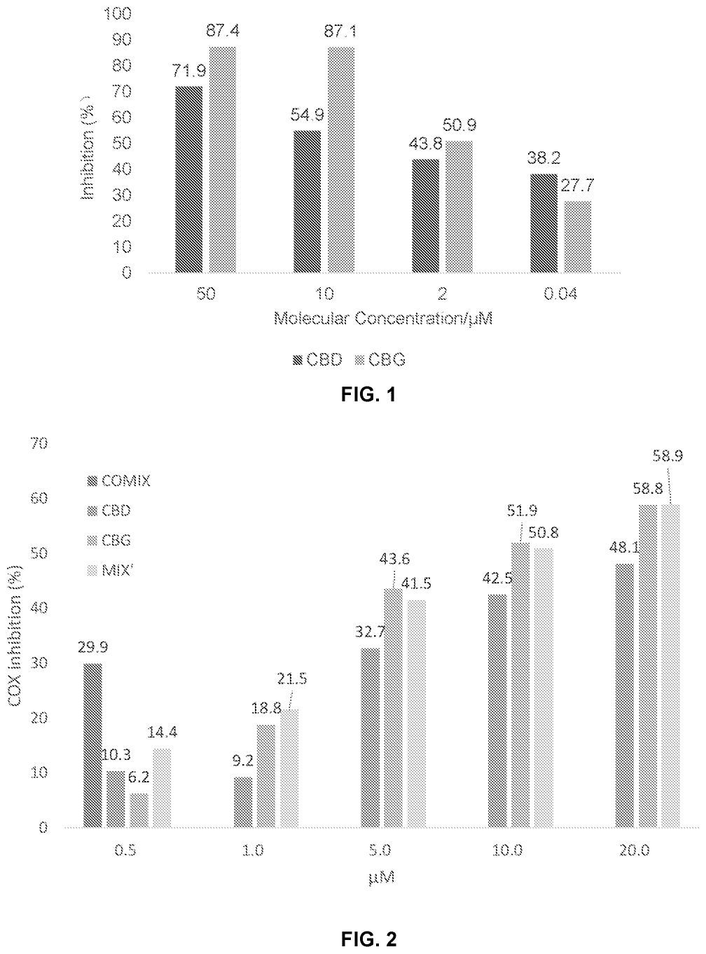

In some embodiments, the composition may have an anti-inflammatory effect by containing CBD, CBG, and BAK. Accordingly, the composition may have the function of treating/repairing skin acne. For example, the composition may have an inhibitory activity/effect against enzymes including inducible nitric oxide synthase (iNOS) and cyclooxygenase-2 (COX-2). For instance, the composition may have an inhibitory activity against COX-2 (also referred to as a COX-2 inhibitory activity/effect) in vitro at multiple total CBD/CBG/BAK molar concentrations (e.g., 5 μM, 10 μM, 12.5 μM, 50 μM, etc.). As another example, the composition may be configured to suppress activity of pro-inflammatory cytokine(s). The pro-inflammatory cytokine(s) may include interleukin 6 (IL-6), interleukin 113 (IL-18), tumor necrosis factor alpha (TNF-α), etc. For instance, the composition may have an ability/function to suppress activity of IL-18 and/or TNF-α at multiple total CBD/CBG/BAK molar concentrations (e.g., 5 μM, 10 μM, 20 μM, etc.). As still another example, the composition may reduce the level of pro-inflammatory mediators, e.g., nitric oxide (NO) and prostaglandin (PGE2). More descriptions regarding the anti-inflammatory effect of the composition may be found elsewhere in the present disclosure (e.g., FIGS. 1-9).

In some embodiments, each of CBD, CBG, and BAK may have an anti-inflammatory effect alone (e.g., at multiple molar concentrations). By combining and/or mixing the CBD, CBG, and/or BAK in the composition, the composition may have a synergistic effect (e.g., a synergistic anti-inflammatory effect) at first total CBD/CBG/BAK molar concentration(s). As used herein, the synergistic anti-inflammatory effect may refer to that is the result of two or more compounds interacting (e.g., mixing or combining) together to produce an anti-inflammatory effect that is greater than the cumulative effect that those compounds produce when used individually. The synergistic anti-inflammatory effect may be measured by inhibitory effect against COX-2 in vitro. The first total CBD/CBG/BAK molar concentration(s) may be near a target total CBD/CBG/BAK molar concentration of the composition, e.g., a difference between a first total CBD/CBG/BAK molar concentration and the target total CBD/CBG/BAK molar concentration may be less than a threshold (e.g, 0.5 μM, 1 μM, 2 μM, etc.). For example, the target total CBD/CBG/BAK molar concentration of the composition may be 10 μM, and the threshold may be 1 μM. The first total CBD/CBG/BAK molar concentration(s) of the composition may be within a range of 9 μM to 11 μM.

In some embodiments, the composition may have an additive anti-inflammatory effect at second total CBD/CBG/BAK molar concentration(s). As used herein, the additive effect may refer to that is the result of two or more compounds interacting together to produce an anti-inflammatory effect that is equal to the cumulative effect that those compounds produce when used individually. The additive anti-inflammatory effect may be measured by inhibitory effect against COX-2 in vitro. The second total CBD/CBG/BAK molar concentration(s) may be greater than the first total CBD/CBG/BAK molar concentration(s). For example, when the first total CBD/CBG/BAK molar concentration(s) of the composition may range from 9 μM to 11 μM, the second total CBD/CBG/BAK molar concentration(s) may be greater than 11 μM, e.g., within a range of 11 μM to 50 μM. For instance, the second total CBD/CBG/BAK molar concentration(s) may be 12.5 μM, 25 μM, 50 μM, etc.

In some embodiments, the composition may have a competitive effect (also referred to as an antagonism effect) at third total CBD/CBG/BAK molar concentration(s). As used herein, the competitive effect may refer to that is the result of two or more compounds interacting together to produce an anti-inflammatory effect that is less than the cumulative effect that those compounds produce when used individually. The competitive anti-inflammatory effect may be measured by inhibitory effect against COX-2 in vitro. The third total CBD/CBG/BAK molar concentration(s) may be less than the first total CBD/CBG/BAK molar concentration(s). For example, when the first total CBD/CBG/BAK molar concentration(s) of the composition may range from 9 μM to 11 μM, the third total CBD/CBG/BAK molar concentration(s) may be less than 9 μM, e.g., within a range of 5 μM to 9 μM. In some embodiments, the third total CBD/CBG/BAK molar concentration(s) of the composition may be less than 5 μM. For example, the third total CBD/CBG/BAK molar concentration(s) of the composition may be 2 μM, 3 μM, etc.

In some embodiments, whether the composition at a specific total CBD/CBG/BAK molar concentration has a synergistic anti-inflammatory effect, an additive anti-inflammatory effect or a competitive anti-inflammatory effect may be determined based on a combination index (CI) of CBD, CBG, and BAK in the composition at the specific total CBD/CBG/BAK molar concentration. The CI of CBD, CBG, and BAK in the composition may also be referred to as a CI of the composition for brevity. If the CI of CBD, CBG, and BAK in the composition at the specific total CBD/CBG/BAK molar concentration is less than 1 (i.e., CI<1), the composition may have a synergistic anti-inflammatory effect at the specific total CBD/CBG/BAK molar concentration. If the CI of CBD, CBG, and BAK in the composition at the specific total CBD/CBG/BAK molar concentration is equal to 1 (i.e., CI=1), the composition may have an anti-inflammatory additive effect at the specific total CBD/CBG/BAK molar concentration. If the CI of CBD, CBG, and BAK in the composition at the specific total CBD/CBG/BAK molar concentration is greater than 1 (i.e., CI>1), the composition may have a competitive anti-inflammatory effect at the specific total CBD/CBG/BAK molar concentration. In some embodiments, the CI of CBD, CBG, and BAK in the composition at the specific total CBD/CBG/BAK molar concentration may be determined by using a CompuSyn software, details of which can be found in “Chou TC. 2010. Drug combination studies and their synergy quantification using the Chou-Talalay method. Cancer Res. 70(2):440-446.”

FIG. 1 is a bar diagram illustrating COX-2 inhibitory effects of CBD and CBG used individually at different molar concentrations according to some embodiments of the present disclosure. Table 1 illustrates COX-2 inhibitory effects of some skincare active components used individually according to some embodiments of the present disclosure. The COX-2 inhibitory effects in FIG. 1 and Table 1 were determined using a fluorometric COX-2 inhibition kit from Cayman Chemical Company (Ann Arbor, Michigan, USA) according to the method of COX-2 inhibition assay as described elsewhere in the present disclosure. The skincare active components in Table 1 include compounds (NMN (i.e., nicotinamide mononucleotide), BAK, resveratrol, caffeine) at 50 μM and extracts (AGE Defense (i.e, Juglans Regia (Walnut) seed extract), RC Bio (i.e., Fagus Sylvatica extract), grape seeds extract, and coffee extract) at 50 μg/mL. As shown in FIG. 1, CBD and CBG used individually had a COX-2 inhibitory effect at different molar concentrations (e.g., 50 μM, 10 μM, even 2 μM, 0.04 μM, etc.), indicating that CBD and CBG had an anti-inflammatory effect when used individually. At the molar concentration of 50 μM, CBD had a COX-2 inhibition of 71.9% and CBG had a COX-2 inhibition of 87.4%. In connection with Table 1, CBG had an optimal COX-2 inhibitory effect among the samples, and CBD had a moderate to upper inhibitory effect among the samples, indicating that CBD and CBG had good anti-inflammatory activity when used individually.

TABLE 1
COX-2 inhibitory effects of some skincare active components

| Sample | Inhibition (%) |
|---|---|
| NMN | 19.63 |
| BAK | 81.87 |
| AGE Defense | 73.60 |
| RC Bio | 77.36 |
| Grape seeds extract | 33.72 |
| Resveratrol | 51.91 |
| Coffee extract | 44.97 |
| Caffeine | 63.53 |

Table 2 illustrates COX-2 inhibitory effects of exemplary compositions of CBD and CBG according to some embodiments of the present disclosure. The COX-2 inhibitory effects in Table 2 were determined using a fluorometric COX-2 inhibition kit from Cayman Chemical Company (Ann Arbor, Michigan, USA) according to the method of COX-2 inhibition assay as described elsewhere in the present disclosure. The compositions A, B, and C in Table 2 may be at 10 μM.

TABLE 2
COX-2 inhibitory effects of compositions of CBD and CBG

| Composition | CBD:CBG | Inhibition/% |
|---|---|---|
| A | 2:1 | 19.63 |
| B | 3:1 | 81.87 |
| C | 5:1 | 73.60 |

In Table 2, a molar concentration of CBD in the composition A was 6.67 μM, and a molar concentration of CBG in the concentration A was 3.33 μM; a molar concentration of CBD in the composition B was 7.5 μM, and a molar concentration of CBG in the concentration B was 2.5 μM; and a molar concentration of CBD in the composition C was 8.3 μM, and a molar concentration of CBG in the concentration C was 1.7 μM. As shown in Table 2, each of compositions A, B, and C had a COX-2 inhibitory effect, indicating that CBD and CBG had an anti-inflammatory when used in combination. In addition, compositions at a certain molar concentration (e.g., 10 μM) with different molar concentration ratios of CBD and CBG therein had different levels of COX-2 inhibitory effect.

FIG. 2 is a bar diagram illustrating COX-2 inhibitory effects of CBD and CBG used individually and in combination at different molar concentrations according to some embodiments of the present disclosure. The COX-2 inhibitory effects in FIG. 1 and Table 1 were determined using a fluorometric COX-2 inhibition kit from Cayman Chemical Company (Ann Arbor, Michigan, USA) according to the method of COX-2 inhibition assay as described in the present disclosure. In FIG. 2. COMIX represents a composition (or mixture) of CBD, CBG, NMN, BAK, AGE Defense, and RC Bio. Concentrations of CBD, CBG, NMN, BAK, AGE Defense, and RC Bio in COMIX were 10 ug/mL, 2 ug/mL, 2 ug/mL, 2 ug/mL, 2 ug/mL, and 2 ug/mL, respectively. The COX-2 inhibitory effect of COMIX was measured for providing an assay basis for designing subsequent COX-2 inhibition assays. MIX′ represents a composition (or mixture) of CBD and CBG at a molar concentration ratio of 1:1. As shown in FIG. 2, CBD and CBG had COX-2 inhibitory effects when used individually and in combination at different molar concentrations. When a total CBD/CBG molar concentration of MIX′ was 0.5 μM, molar concentrations of CBD and CBG in MIX′ were 0.25 μM. Similarly, when a total CBD/CBG molar concentration of MIX′ was 1.0 μM, molar concentrations of CBD and CBG in MIX′were 0.5 μM; when a total CBD/CBG molar concentration of MIX′ was 5 μM, molar concentrations of CBD and CBG in MIX were 2.5 μM; when a total CBD/CBG molar concentration of MIX′ was 10 μM, molar concentrations of CBD and CBG in MIX′ were 5 μM; and when a total CBD/CBG molar concentration of MIX′ was 20 μM, molar concentrations of CBD and CBG in MIX′ were 10.0 μM. MIX′ with a same molar concentration ratio of CBD and CBG (e.g., 1:1) at different total CBD/CBG molar concentrations had different levels of COX-2 inhibitory effect.

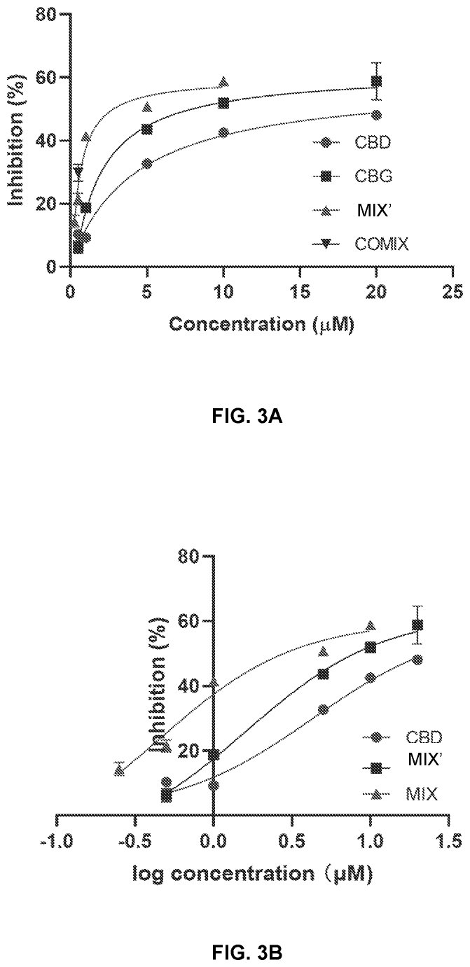

FIGS. 3A and 3B are curve diagrams illustrating COX-2 inhibitory effects of CBD and CBG used individually and in combination at different molar concentrations corresponding to FIG. 2 according to some embodiments of the present disclosure. In FIG. 3A, the horizontal axis represents molar concentrations, and the vertical axis represents COX-2 inhibitions; and in FIG. 3B, the horizontal axis represents log molar concentrations (i.e., the logarithm of the molar concentrations in FIG. 3A), and the vertical axis represents COX-2 inhibitions. The curve diagram of FIG. 3A may also be referred to as a dose-response curve. According to curves in FIGS. 3A and 3B, COX-2 inhibitory effects of CBD, CBG, and MIX′ reached a plateau after molar concentrations thereof exceeded 10 μM. Inhibitions corresponding to plateau were about 60%. During the plateau, inhibitory effects of CBD, CBG, and MIX′ would not increase as the molar concentration increases.

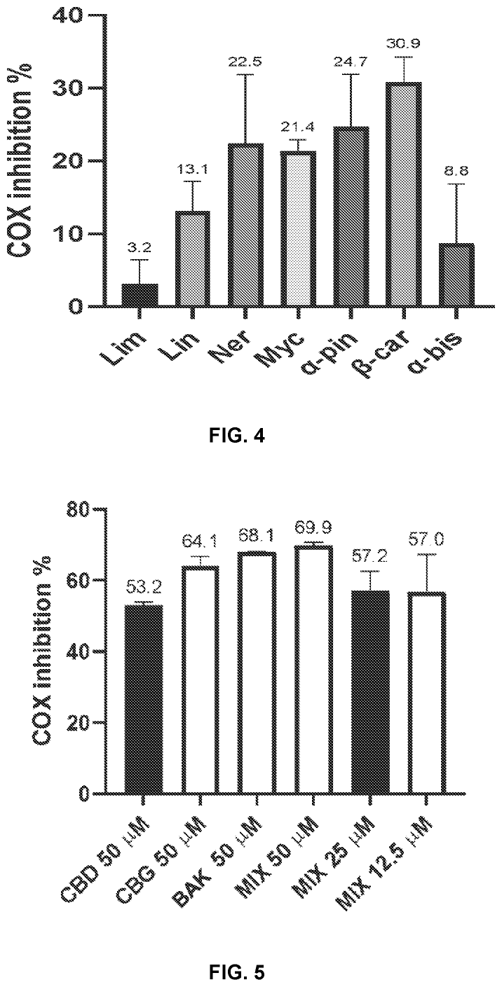

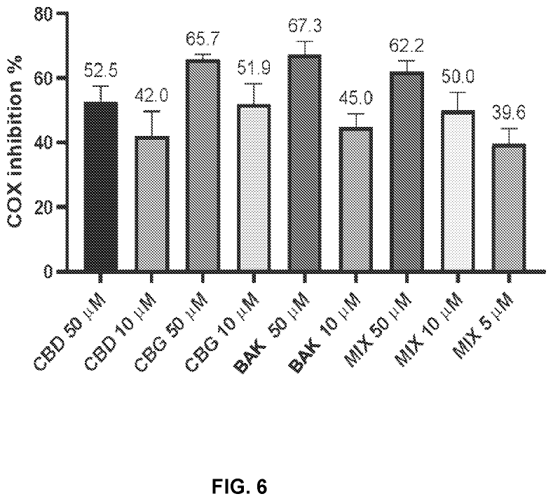

FIG. 4 is a bar diagram illustrating COX-2 inhibitory effects of some personal care ingredients used individually at 50 μM according to some embodiments of the present disclosure. FIGS. 5 and 6 are bar diagrams illustrating COX-2 inhibitory effects of CBD, CBG, and BAK used individually and in combination at different molar concentrations according to some embodiments of the present disclosure. The COX-2 inhibitory effects in FIGS. 4-6 were determined using a fluorometric COX-2 inhibition kit from BioVision Inc. (Mountain View, CA, USA) according to the methods described in COX-2 inhibition assay. The personal care ingredients Lim, Lin, Ner, Myc, α-pin, β-car, and α-bis represent limonene, linalool, nerolidol, myrcene, alpha-pinene, beta-caryophyllene, and alpha-bisabolol, respectively. In FIGS. 5 and 6, MIX represents a composition (or mixture) of CBD, CBG, and BAK at a molar concentration ratio of 1:1:1. MIX 50 μM, MIX 25 μM, and 12.5 μM represent total CBD/CBG/BAK molar concentrations of MIX being 50 μM, 25 μM, and 12.5 μM, respectively. CBD 50 μM represents that CBD is used individually and a molar concentration of CBD is 50 μM. Similarly, CBG 50 μM represents that CBG is used individually and a molar concentration of CBG is 50 μM, and BAK 50 μM represents that BAK is used individually and a molar concentration of BAK is 50 μM According to FIGS. 5 and 6, CBD, CBG, BAK, and MIX at 50 μM had better COX-2 inhibitory effects than the personal care ingredients at 50 μM. As shown in FIG. COX-2 inhibitions of CBD, CBG, BAK, and MIX′ at 12.5 μM, 25 μM, and 50 μM were all around 60%, indicating that CBD, CBG, BAK, and MIX′ were under the plateau at 12.5 μM, 25 μM, and 50 μM as described in connection with FIGS. 3A and 3B.

Further as shown in FIG. 6, COX-2 inhibitory effects of MIX including CBD, CBG, and BAK were measured at two total CBD/CBG/BAK molar concentrations (10 μM and 50 μM). At the higher concentration (i.e., 50 μM), CBD, CBG, and BAK had COX-2 inhibitory activities of 52.5%, 65.7%, and 67.3%, respectively. At the lower concentration (i.e., 10 μM), CBD, CBG, and BAK had COX-2 inhibitory activities of 42.0%, 51.9%, 45.0%, respectively. MIX of CBD, CBG, and BAK at a molar concentration ratio of 1:1:1 had COX-2 inhibitory activities of 39.6%, 50.0%, and 62.2%, at molar concentrations of 5 μM, 10 μM, and 50 μM, respectively. In some embodiments, resveratrol was used as a positive control which had a COX-2 inhibitory activity of 50.1% at a molar concentration of 20 μM (which is not shown in FIG. 6) for determining the COX-2 inhibitory effects of CBD, CBG, BAK, and MIX.

In some embodiments, CIs of MIX of CBD, CBG, and BAK at different molar concentrations were determined using the CompuSyn software as described elsewhere in the present disclosure. For example, the CompuSyn software can be used to predict/estimate CIs of the MIX at any total CBD/CBG/BAK molar concentrations based on two sets of inhibitions of CBD, CBG, BAK, and the MIX at two molar concentrations. As shown in Table 3, the CI of MIX at 10 μM was 0.74, suggesting that MIX of CBD, CBG, and BAK had a synergistic anti-inflammatory effect on the COX-2 inhibition at 10 μM. The CI of MIX at 50 μM was 1.0, suggesting that MIX of CBD, CBG, and BAK had an additive anti-inflammatory effect on the COX-2 inhibition at 50 μM. The CI of MIX at 2 μM was 1.16, suggesting that MIX of CBD, CBG, and BAK had a competitive anti-inflammatory effect on the COX-2 inhibition at 2 μM. The CIs of MIX at 50 μM, 10 μM, 2 μM were determined based on inhibitions of CBD, CBG, CBK, and the MIX at 50 μM and inhibitions of CBD, CBG, CBK, and the MIX at 10 μM using the CompuSyn software.

TABLE 3
Inhibition Combination Indexes (CIs) of
MIX at different molar concentrations

| Sample | Inhibition (%) | | |
|---|---|---|---|
| Molar concentration | 50 μM | 10 μM | 2 μM |
| CBD | 52.5 | 42.0 | — |
| CBG | 65.7 | 51.9 | — |
| BAK | 67.3 | 45.0 | — |
| MIX | 62.2 | 50.0 | — |
| Combination Index (CI) | 1.0 | 0.74 | 1.16 |

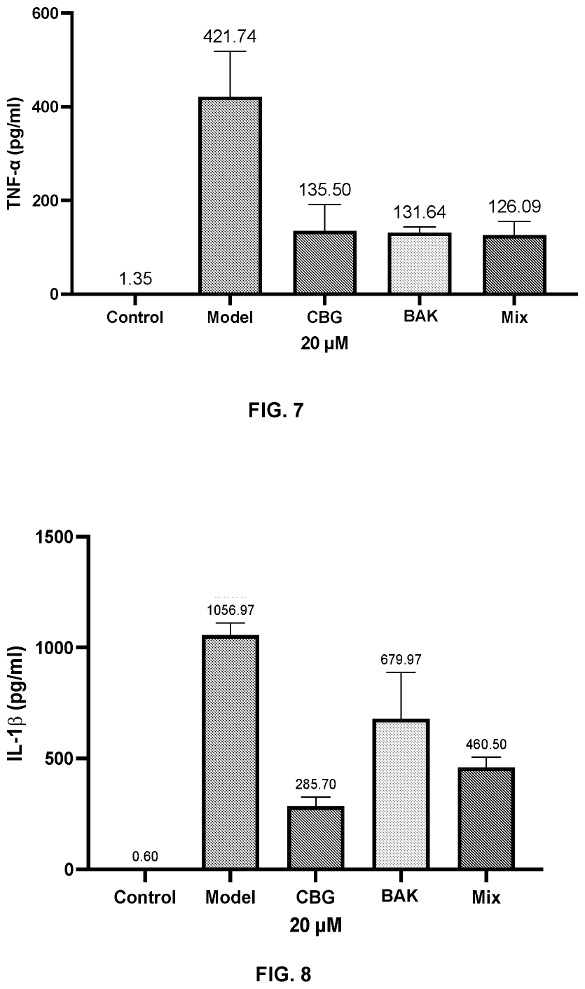

FIGS. 7 and 8 are bar diagrams illustrating exemplary abilities of CBG, BAK, and a composition (i.e., Mix) of CBD, CBG, and BAK at a molar concentration of 1:1:1 to suppress activities of pro-inflammatory cytokines according to some embodiments of the present disclosure. In FIGS. 7 and 8, Model represents an inflammatory model that was induced by lipopolysaccharides and nigericin. The abilities/functions of CBG, BAK, and Mix to suppress pro-inflammatory cytokines in FIGS. 7 and 8 were measured at a total CBD/CBG/BAK molar concentration of 20 μM. An ability/function to suppress activity of a pro-inflammatory cytokine was reflected by a remaining concentration of the pro-inflammatory cytokine after the reaction. The greater the remaining concentration of the pro-inflammatory cytokine, the worse the ability to suppress activity of the pro-inflammatory cytokine. As shown in FIG. 7, remaining concentrations of TNF-α corresponding to CBG, BAK, and Mix at the total CBD/CBG/BAK molar concentration of 20 μM were lower than the remaining concentration of TNF-α corresponding to Model, reflecting that CBG, BAK, and Mix at the total CBD/CBG/BAK molar concentration of 20 μM can suppress activity of TNF-α in cell level. As shown in FIG. 8, remaining concentrations of IL-113 corresponding to CBG, BAK CBG, BAK, and Mix at 20 μM were lower than the remaining concentration of IL-1(3 corresponding to Model, reflecting that CBG, BAK, and Mix at 20 μM had an activity to suppress activity of IL-113 in cell level. In connection with FIGS. 7 and 8, CBD, CBG, and Mix at 20 μM were indicated to have an anti-inflammatory effect.

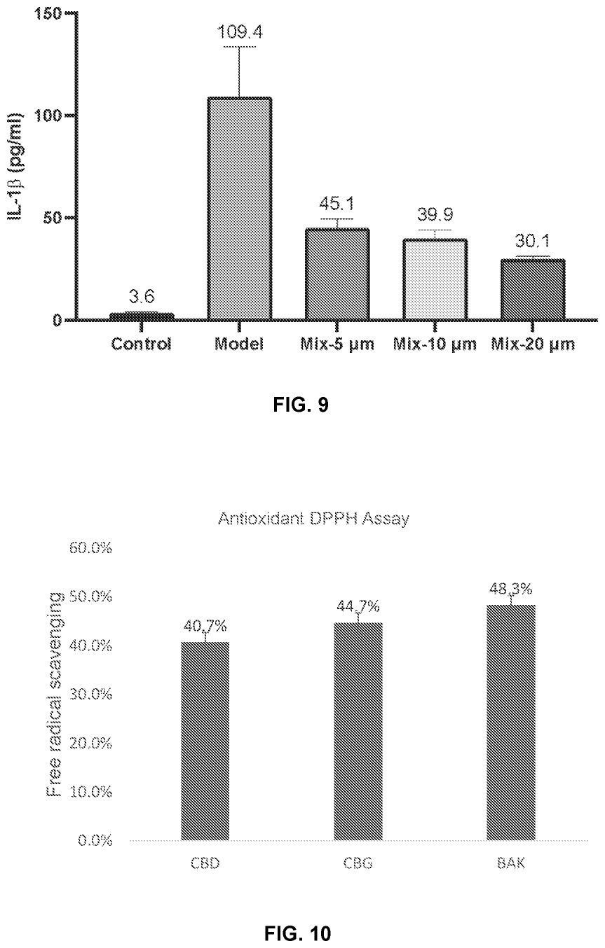

FIG. 9 is a bar diagram illustrating exemplary abilities of a composition (i.e., Mix) of CBD, CBG, and BAK at a molar concentration of 1:1:1 at different molar concentrations to suppress activity of IL-113 according to some embodiments of the present disclosure. In FIG. 9, Model represents an inflammatory model which was different from that in FIG. 8. A difference between remaining concentrations (109.4 pg/mL and 3.6 pg/mL) of IL-1p corresponding to Control and Model in FIG. 9 was 105.8 pg/mL, which was less than a difference (1050.37 pg/m L) between remaining concentrations (1050.97 pg/mL and 0.6 pg/m L) of IL-1p corresponding to Control and Model in FIG. 8. As shown in FIG. 9, remaining concentrations of IL-1p corresponding to Mix at different total CBD/CBG/BAK molar concentrations (e.g., 5 μM, 10 μM, and 20 μM) were lower than the remaining concentration of IL-1p corresponding to Model, reflecting that Mix at different total CBD/CBG/BAK molar concentrations can suppress activity of IL-1p in cell level, indicating that Mix had an anti-inflammatory effect at different total CBD/CBG/BAK molar concentrations. In addition, the higher the molar total CBD/CBG/BAK concentration of Mix, the higher the ability to suppress activity of IL-1p of Mix.

In some embodiments, the composition may have an antioxidant activity/effect. FIG. 10 is a bar diagram illustrating antioxidant effects of CBD, CBG, and BAK used individually according to some embodiments of the present disclosure. In some embodiments, data in the bar diagram in FIG. 10 was determined (e.g., measured) according to an antioxidant DPPH (i.e., α, α-diphenyl-β-picrylhydrazyl) assay that was well known for the skills in the art. An antioxidant effect was reflected by free radical scavenging after the reaction. The greater the free radical scavenging, the better the antioxidant effect. As shown in FIG. 10, CBD, CBG, and BAK had an antioxidant effect when used individually. In addition, BAK had a better antioxidant effect than CBG and CBG.

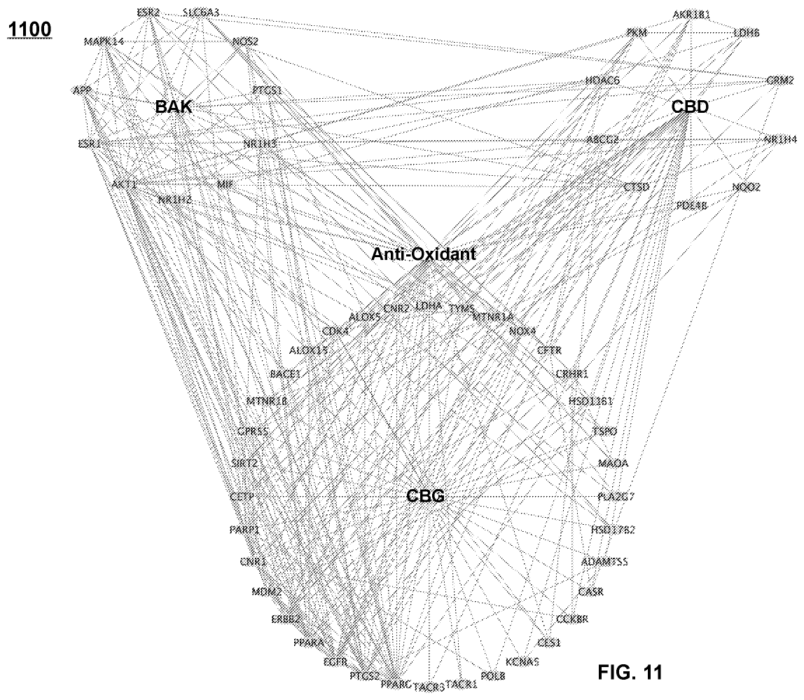

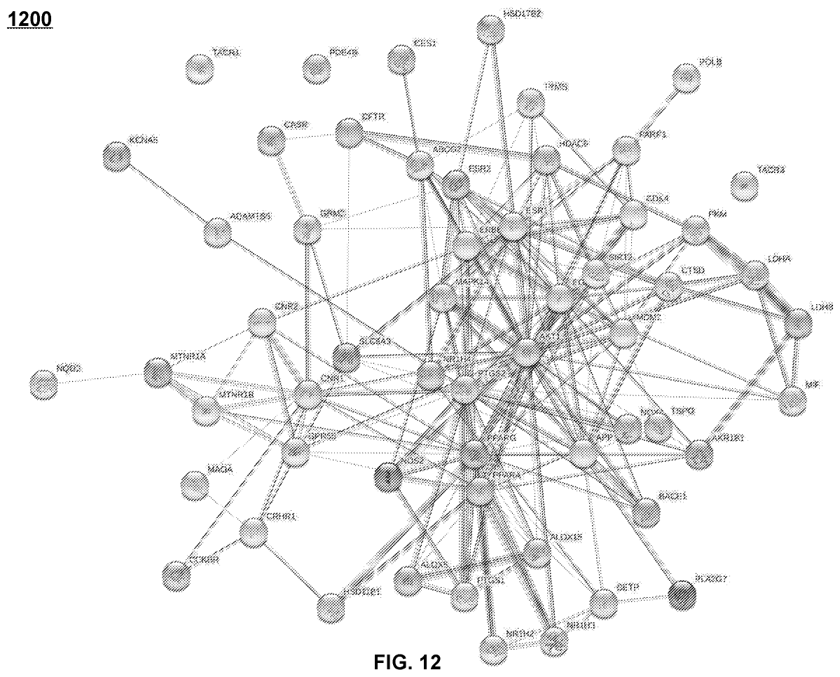

FIG. 11 is a diagram illustrating an exemplary target network of CBD, CBG, and BAK according to some embodiments of the present disclosure. FIG. 12 is a diagram illustrating an exemplary protein-protein interaction (PPI) network according to some embodiments of the present disclosure. In some embodiments, the target network 1100 was generated based on a GeneCards database, a therapeutic target database (TTD), a DisGeNET database, or the like, or any combination thereof. The target network 1100 showed potential antioxidant targets related to each of the CBD, CBG, and BAK. The PPI network 1200 was generated based on the target network 1100 (e.g., using a Cytoscape 3.7.2 software). The PPI network 1200 was related to potential antioxidant targets of a combination of CBD, CBG, and BAK. According to parameters (e.g., a betweenness centrality, a closeness centrality, a node degree, etc.) of the PPI network 1200, key antioxidant targets of the combination of CBD, CBG, and BAK were determined and were illustrated in Table 4, indicating that the composition of CBD, CBG, and BAK had an antioxidant effect and multiple antioxidant targets participated in/contributed to the antioxidant effect of the combination of CBD, CBG, and BAK.

TABLE 4
Key antioxidant targets of the combination
of CBD, CBG, and BAK

| Target | Betweenness Centrality | Closeness Centrality | Node Degree |
|---|---|---|---|
| AKT1 | 0.070107 | 0.674157 | 34 |
| PPARG | 0.038432 | 0.638298 | 26 |
| PTGS2 | 0.032589 | 0.625 | 24 |
| EGFR | 0.021983 | 0.618557 | 23 |
| ESR1 | 0.018086 | 0.576923 | 19 |
| PPARA | 0.016246 | 0.588235 | 18 |
| ERBB2 | 0.009816 | 0.582524 | 17 |
| APP | 0.012215 | 0.550459 | 16 |
| MDM2 | 0.008368 | 0.576923 | 16 |
| CNR1 | 0.018419 | 0.571429 | 15 |
| MAPK14 | 0.006525 | 0.540541 | 14 |
| PARP1 | 0.007332 | 0.555556 | 12 |
| ESR2 | 0.005858 | 0.530973 | 12 |
| SLC6A3 | 0.010386 | 0.530973 | 11 |
| CETP | 0.008633 | 0.545455 | 10 |
| GPR55 | 0.002031 | 0.545455 | 10 |
| SIRT2 | 0.0026 | 0.545455 | 10 |
| CTSD | 0.003272 | 0.545455 | 10 |
| ABCG2 | 0.004075 | 0.545455 | 10 |
| ALOX15 | 0.002538 | 0.540541 | 9 |
| ALOX5 | 0.001796 | 0.540541 | 9 |
| HDAC6 | 0.003082 | 0.517241 | 9 |
| BACE1 | 0.001794 | 0.540541 | 9 |
| CDK4 | 0.00104 | 0.540541 | 9 |
| CNR2 | 0.000968 | 0.530973 | 8 |
| LDHA | 0.003142 | 0.530973 | 8 |
| PTGS1 | 0.000716 | 0.504202 | 8 |
| NOS2 | 0.000536 | 0.504202 | 8 |
| PKM | 0.001573 | 0.530973 | 8 |
| AKR1B1 | 0.001236 | 0.530973 | 8 |

Further, Table 5 illustrates potential antioxidant pathways of the combination of CBD, CBG, and BAK according to some embodiments of the present disclosure. Data in Table 5 was generated using a Cytoscape software (e.g., the Cytoscape 3.7.2 software). As shown in Table, 5, for a specific category of a pathway, the greater the GeneCount corresponding to the pathway, the more relevant the pathway to the antioxidant effect of the composition of CBD, CBG, and BAK.

TABLE 5
Potential antioxidant pathways of the combination of CBD, CBG, and BAK

| Category | Term | GeneCount | % | P-Value |
|---|---|---|---|---|
| GOTERM_BP_DIRECT | cannabinoid signaling pathway | 3 | 9.7 | 9.2E−06 |
| GOTERM_BP_DIRECT | response to drug | 7 | 22.6 | 0.000014 |
| GOTERM_BP_DIRECT | response to oxidative stress | 5 | 16.1 | 0.000042 |
| GOTERM_BP_DIRECT | positive regulation of nitric oxide | 4 | 12.9 | 0.000061 |
| | biosynthetic process | | | |
| GOTERM_BP_DIRECT | positive regulation of smooth | 4 | 12.9 | 0.00016 |
| | muscle cell proliferation | | | |
| GOTERM_BP_DIRECT | lipoxygenase pathway | 3 | 9.7 | 0.00024 |
| GOTERM_BP_DIRECT | negative regulation of macrophage | 3 | 9.7 | 0.00024 |
| | derived foam cell differentiation | | | |
| GOTERM_BP_DIRECT | positive regulation of transcription | 9 | 29 | 0.00024 |
| | from RNA polymerase II promoter | | | |
| GOTERM_BP_DIRECT | response to nutrient | 4 | 12.9 | 0.00031 |
| GOTERM_BP_DIRECT | circadian rhythm | 4 | 12.9 | 0.00032 |
| GOTERM_BP_DIRECT | wound healing | 4 | 12.9 | 0.00039 |
| GOTERM_BP_DIRECT | oxidation-reduction process | 7 | 22.6 | 0.00054 |
| GOTERM_BP_DIRECT | cellular response to hypoxia | 4 | 12.9 | 0.00066 |
| GOTERM_BP_DIRECT | glucose homeostasis | 4 | 12.9 | 0.00076 |
| GOTERM_BP_DIRECT | positive regulation of sequence- | 4 | 12.9 | 0.00085 |
| | specific DNA binding | | | |
| | transcription factor activity | | | |
| GOTERM_CC_DIRECT | perinuclear region of cytoplasm | 9 | 29 | 5.20E−06 |
| GOTERM_CC_DIRECT | membrane raft | 6 | 19.4 | 2.00E−05 |
| GOTERM_CC_DIRECT | axon | 5 | 16.1 | 4.60E−04 |
| GOTERM_CC_DIRECT | nucleus | 19 | 61.3 | 5.50E−04 |
| GOTERM_CC_DIRECT | plasma membrane | 16 | 51.6 | 9.20E−04 |
| GOTERM_CC_DIRECT | cytosol | 14 | 45.2 | 1.30E−03 |
| GOTERM_CC_DIRECT | protein complex | 5 | 16.1 | 4.40E−03 |
| GOTERM_CC_DIRECT | paranodal junction | 2 | 6.5 | 8.20E−03 |
| GOTERM_CC_DIRECT | cytoplasm | 16 | 51.6 | 1.10E−02 |
| GOTERM_CC_DIRECT | nuclear envelope lumen | 2 | 6.5 | 1.30E−02 |
| GOTERM_MF_DIRECT | enzyme binding | 12 | 38.7 | 5.80E−12 |
| GOTERM_MF_DIRECT | cannabinoid receptor activity | 3 | 9.7 | 9.10E−06 |
| GOTERM_MF_DIRECT | RNA polymerase II transcription | 4 | 12.9 | 3.50E−05 |
| | factor activity, ligand-activated | | | |
| | sequence-specific DNA binding | | | |
| GOTERM_MF_DIRECT | identical protein binding | 9 | 29 | 3.50E−05 |
| GOTERM_MF_DIRECT | nitric-oxide synthase regulator | 3 | 9.7 | 8.50E−05 |
| | activity | | | |
| GOTERM_MF_DIRECT | steroid hormone receptor activity | 4 | 12.9 | 1.30E−04 |
| GOTERM_MF_DIRECT | protein phosphatase binding | 4 | 12.9 | 1.90E−04 |
| GOTERM_MF_DIRECT | drug binding | 4 | 12.9 | 3.30E−04 |
| GOTERM_MF_DIRECT | protein binding | 25 | 80.6 | 1.50E−03 |
| GOTERM_MF_DIRECT | transcription factor binding | 5 | 16.1 | 1.50E−03 |

In some embodiments, the composition may include a variety of optional ingredients that are known for use in personal care composition, as long as the optional ingredient(s) do not unduly alter product stability, aesthetics, or performance. The optional ingredients, when incorporated into the composition, should be suitable for contact with human keratinous tissue without undue toxicity, incompatibility, instability, allergic response, and the like within the scope of sound judgment. The optional ingredients may include abrasives, absorbents, opacifying agents, colorings/colorants (e.g., pigments, dyes, and lakes), particles, essential oils, anti-caking agents, foaming agents, anti-foaming agents, oil control agents, binders, biological additives, vitamins, minerals, peptides, sugar amines, flavonoid compounds, anti-oxidants, preservatives, phytosterols, protease inhibitors, tyrosinase inhibitors, exfoliating agents, skin lightening agents, sunless tanning agents, thickeners, pH adjusters, anti-acne active components, anti-cellulite active components, anti-wrinkle active components, phytosterols and/or plant hormones, N-acyl amino acid compounds, antimicrobials, antifungals, moisturizers, emollients, humectants, lubricating agents, fragrances, anti-dandruff agents, buffering agents, bulking agents, chelating agents, biocides, denaturants, astringents, external analgesics, anti-inflammatory agents, sunscreen agents, film formers and/or polymers for aiding film-forming properties and substantivity of the composition, propellants, reducing agents, sequestrants, conditioning agents, or the like, or any combination thereof. For example, the optional ingredient(s) of the composition may include vitamins, proteins, zeolites, peptides, skin-lightening, sunscreen active components, terpene alcohols, desquamation active components, anti-acne active components, anti-wrinkle active components, anti-atrophy active components, anti-oxidants, flavonoids, anti-inflammatory agents, anti-cellulite agents, tanning active components, skin-soothing active components, skin healing active components, conditioning agents, or the like, or any combination thereof. The anti-acne active components refer to active components for treating acne (e.g., hormonal acne), removing blackheads, and/or repairing acne scars, hyperpigmentation, skin redness spots, etc. For instance, the anti-acne active components may include a hydroxy acid, an additional cannabinoid besides CBD and CBG (or a derivative thereof), a cannaflavin, or the like, or any combination thereof. The hydroxy acid may include an alpha hydroxy acid, a beta hydroxy acid, or the like, or any combination thereof. The additional cannabinoid may include cannabichromene (CBC), tetrahydrocannabinol (THC), cannabidiolic acid (CBDA), cannabigerolic acid (CBGA), tetrahydrocannabivarin (THCV), or the like, or any combination thereof. The cannaflavin may include cannaflavin A, cannaflavin B, or the like, or any combination thereof.

In some embodiments, the composition may further include a pharmaceutically and/or dermatologically acceptable carrier. For instance, the carrier may include a coating layer, a capsule, a microcapsule, a nanocapsule, or the like, or any combination thereof. It should be noted that the carrier may need to be non-toxic and may not have significant impacts on the activity of the key components (e.g., CBD/CBG/BAK) in the composition. In some embodiments, the carrier may provide protection for the key components against some undesired conditions, such as oxidation, the decomposition, or inactivation of the key components. For instance, the carrier may help maintain or increase the efficacy of the composition by protecting the key components in the composition. In some embodiments, the carrier may be used for the controlled release of the key components. The controlled release may include but is not limited to slow release, sustained release, targeted release, or the like. For instance, the carrier may include Nano lipids, oil-in-water emulsions, hydrogel capsules, microcapsules or nanocapsules made of collagen, gelatin, chitosan, alginate, polyvinyl alcohol, polyethylene oxide, starch, cross-linked starch, or the like, or any combination thereof. In some embodiments, the carrier may facilitate a controlled release of the key components in the pharmaceutical composition.

In some embodiments, the composition may be formulated as a tablet, capsule, granules, powder, micelles, liquid, suspension, cream, foam, gels, lotion, pastes, or ointment.

In some embodiments, resulting from the anti-inflammatory and/or antioxidant effects that the composition of CBD, CBG, and BAK possess as described elsewhere in the present disclosure, the composition may be used or applied to treat inflammation in a subject. The inflammation in a subject may include a chronic inflammation. The chronic inflammation may have symptoms including a skin rash. (e.g., repeated acne or psoriasis), an abdominal pain, a chest pain, fatigue (e.g., systemic lupus), a fever (e.g., tuberculosis), joint pain or stiffness (e.g., rheumatoid arthritis), a mouth sores (e.g., HIV infection), or the like, or any combination thereof. In some embodiments, the chronic inflammation may be caused by autoimmune disorders (e.g., lupus, where the subject's body attacks healthy tissue), exposure to toxins (e.g., pollution or industrial chemicals), an untreated acute inflammation (e.g., from an infection or injury), drinking alcohol in excess, having a high body mass index (BMI) that falls within the ranges for obesity, exercise at a maximum intensity too frequently (or not exercising enough), experiencing chronic stress, smoking, or the like, or any combination thereof.

In some embodiments, the method for treating inflammation in the subject may include administering the composition as described elsewhere in the present disclosure to the subject suffering from a disease related to inflammation (e.g., chronic inflammation). For example, the composition may be administered to the subject via an oral administration, an injection administration, a topical administration (e.g., a localized application), or the like, or any combination thereof. In some embodiments, the injection administration may include subcutaneous injection, intramuscular injection, intravenous injection, or the like. For example, the injection administration may include injection of the composition into the kidney, liver, heart, thyroid or joints, etc. In some embodiments, the topical administration may include applying the composition on the skin. For example, the topical administration may include vaginal administration, rectal administration, nasal administration, auricular administration, intramedullary administration, intra-articular administration, intra-pleural administration, or the like, or any combination thereof. In some embodiments, the method may include administering the composition to the subject three times a day, two times a day, one time a day, once every two days, etc.

In some embodiments, the present disclosure provides the use of a composition in the manufacture of a medicament for the treatment of inflammation in a subject. The composition may include CBD, CBG, and BAK, a molar concentration ratio of CBD, CBG, and BAK may be 1:1:1 in the composition, and a molar concentration of CBD in the composition may be in a range of 1.67 μM to 16.7 μM. More descriptions regarding the composition may be found elsewhere in the present disclosure. The use of the composition may be the same as or similar to the description of administering the composition to the subject.

For illustration purposes, for the composition used as a personal care composition (e.g., a skincare product), the method of using the composition may include identifying a target portion of keratinous tissue (e.g., a facial skin surface such as the forehead, perioral, chin, periorbital, nose, and/or cheek) in need of treatment/repairment and/or where treatment/repairment is desired; and applying a safe and effective amount of the composition to the target portion of tissue. Without intending to be bound by theory, it is believed that the application of an effective amount of the composition to a target portion of keratinous tissue in need of treatment or where treatment is desired can provide the desired appearance benefit over the course of a treatment period.

The treatment period should be of sufficient time for the component(s) in the composition to provide the desired benefit to the target portion of keratinous tissue (e.g., improve appearance, increase moisturization). The treatment period may last for at least 1 week (e.g., about 2 weeks, 4 weeks, 8 weeks, or even 12 weeks). For example, the treatment period may extend over multiple months (i.e., 3-12 months) or multiple years. As another example, the composition may be applied most days of the week (e.g., at least 4, 5, or 6 days a week), at least once a day, or even twice a day during a treatment period of at least 2 weeks, 4 weeks, 8 weeks, or 12 weeks.

The step of applying the composition may be accomplished by localized application. In reference to the application of the composition, the terms “localized,” “local,” or “locally” may refer to that the composition is delivered to the targeted area (e.g., a hyperpigmented portion of the skin) while minimizing delivery to keratinous surfaces where treatment is not desired. For example, the composition may be applied and lightly massaged into an area of skin. The form of the composition or the dermatologically acceptable carrier should be selected to facilitate localized application. While certain embodiments herein contemplate applying a composition locally to an area, it should be noted that compositions herein may be applied more generally or broadly to one or more skin surfaces.

The composition may be applied in a variety of manners, including by rubbing, wiping, or dabbing with hands or fingers, or by an implement and/or delivery enhancement device. Exemplary implements may include a sponge or sponge-tipped applicator, a swab (for example, a cotton-tipped swab), a pen optionally comprising a foam or sponge applicator, a brush, a wipe, or the like, or any combination thereof. Exemplary delivery enhancement devices may include magnetic, mechanical, electrical, ultrasonic, and/or other energy devices. For example, the composition may be spread onto the skin to facilitate the separation of the aqueous phase from the oil phase. When the aqueous and oil phases have separated, the composition may be left on the keratinous tissue. Alternatively, the composition may be allowed to remain on the skin for 5 seconds, 10 seconds, 30 seconds, or 1 minute before being rubbed into the keratinous tissue.

COX-2 Inhibition Assay

Materials and methods: the inhibitory effects of test samples (e.g., CBD, CBG, BAK, Mix, a positive control, and/or other compounds and compositions described in the present disclosure) against COX-2 were measured by a fluorometric COX-2 inhibition kit from Cayman Chemicals (Ann Arbor, Michigan, USA) or BioVision Inc. (Mountain View, CA, USA) following the manufacturer's instructions. Briefly, a COX-2 enzyme was reconstituted with 110 μL of sterile distilled H₂O and arachidonic acid was dissolved in 55 μL ethanol. COX cofactor was diluted with COX assay buffer for 200 times, and arachidonic acid and NaOH were mixed in a molar ratio of 1:1 before it was diluted for 10 times for further use. COX assay buffer (84 μL), COX probe (1 μL), diluted COX cofactor (2 μL), COX-2 enzyme (1 μL), and test samples (2 μL) were incubated in a 96-well plate at room temperature for 10 min. Next, diluted arachidonic acid/NaOH solution (10 μL) was added to each well of the 96-well plate to initiate the reaction. The fluorescence of each well was recorded at an excitation wavelength of 535 nm and an emission wavelength of 587 nm with a kinetic mode for 10 min using a plate reader (SpectraMax® M2, Molecular Devices Corp., operated by SoftmaxPro® v.4.6 software, Sunnyvale, CA, USA). Resveratrol was used as a positive control. Two points (T1 and T2) in a linear range of a plot related to the recorded fluorescence were used to obtain corresponding fluorescence values (RFU₁ and RFU₂). Then, a slope for a sample was calculated by dividing a net ΔRFU (RFU₂-RFU₁) value by the time ΔT (T2-T1). An inhibition (%) of COX-2 of the sample was determined as the following equation: Inhibition (%)=((slope of control−slope of the sample)/slope of control)×100.

The method of COX-2 inhibition assay is a suitable manner for determining the COX-2 inhibitory effects of samples described elsewhere in the present disclosure, which indicates the anti-inflammatory effect of the samples.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended for those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and “some embodiments” mean that a particular feature, structure, or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures, or characteristics may be combined as suitable in one or more embodiments of the present disclosure.

Furthermore, the recited order of processing elements or sequences, or the use of numbers, letters, or other designations, therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software-only solution, e.g., an installation on an existing server or mobile device.

Similarly, it should be appreciated that in the foregoing description of embodiments of the present disclosure, various features are sometimes grouped together in a single embodiment, figure, or description thereof to streamline the disclosure aiding in the understanding of one or more of the various embodiments. This method of disclosure, however, is not to be interpreted as reflecting an intention that the claimed subject matter requires more features than are expressly recited in each claim. Rather, claim subject matter lies in less than all features of a single foregoing disclosed embodiment.

1. A composition, comprising:
cannabidiol (CBD), cannabigerol (CBG), and bakuchiol (BAK), wherein a molar concentration ratio of CBD, CBG, and BAK is 1:1:1 in the composition, and a molar concentration of CBD in the composition is in a range of 1.67 μM to 16.7 μM.

2. The composition of claim 1, wherein a total CBD/CBG/BAK molar concentration of the composition is in a range of 5 μM to 50 μM, the total CBD/CBG/BAK molar concentration of the composition being defined by a sum of molar concentrations of CBD, CBG, and BAK in the composition.

3. The composition of claim 2, wherein the total CBD/CBG/BAK molar concentration of the composition is 5 μM, 10 μM, 12.5 μM, 25 μM, or 50 μM.

4. The composition of claim 2, wherein the composition has an inhibitory activity against cyclooxygenase (COX)-2 in vitro.

5. The composition of claim 2, wherein the composition has a target total CBD/CBG/BAK molar concentration that exhibits a synergistic anti-inflammatory effect, a combination index of CBD, CBG, and BAK in the composition corresponding to the target-total CBD/CBG/BAK molar concentration being less than 1.

6. (canceled)

7. The composition of claim 5, wherein the target total CBD/CBG/BAK molar concentration of the composition is 10 μM.

8. The composition of claim 1, wherein the composition is configured to suppress activity of a pro-inflammatory cytokine.

9. (canceled)

10. The composition of claim 1, wherein the composition has an antioxidant activity.

11. The composition of claim 1, further comprising an active component for treating acne or repairing acne scars.

12. The composition of claim 11, wherein the active component comprises at least one of a hydroxy acid, an additional cannabinoid, or a cannaflavin.

13. The composition of claim 12, wherein the additional cannabinoid comprises at least one of cannabidiolic acid (CBDA) or cannabigerolic acid (CBGA), or the cannaflavin comprises at least one of cannaflavin A or cannaflavin B.

14-20. (canceled)

21. A composition, comprising:
cannabidiol (CBD), cannabigerol (CBG), and bakuchiol (BAK), wherein
a molar concentration of CBD in the composition is in a range of 1.67 μM to 16.7 μM;
a total CBD/CBG/BAK molar concentration of the composition is in a range of 5 μM to 50 μM, the total CBD/CBG/BAK molar concentration of the composition being defined by a sum of molar concentrations of CBD, CBG, and BAK in the composition; and
the composition has a synergistic anti-inflammatory effect or an additive anti-inflammatory effect.

22. The composition of claim 21, wherein the synergistic anti-inflammatory effect or the additive anti-inflammatory effect is measured by inhibitory effect against COX-2 in vitro.

23. The composition of claim 21, wherein the total CBD/CBG/BAK molar concentration of the composition is in a range of 9 μM to 11 μM.

24. The composition of claim 21, further comprising cannabidiolic acid (CBDA) or cannabigerolic acid (CBGA).

25. The composition of claim 21, wherein a molar concentration ratio of CBD, CBG, and BAK is 1:1:1.

26. A composition, comprising:
cannabidiol (CBD), cannabigerol (CBG), and bakuchiol (BAK), wherein
a molar concentration ratio of CBD, CBG, and BAK is 1:1:1 in the composition;
a molar concentration of CBD in the composition is in a range of 1.67 μM to 16.7 μM;
the composition has a synergistic anti-inflammatory effect at a first total CBD/CBG/BAK molar concentration, a combination index of CBD, CBG, and BAK in the composition corresponding to the first total CBD/CBG/BAK molar concentration being less than 1; and
the composition has an additive anti-inflammatory effect at a second total CBD/CBG/BAK molar concentration, wherein:
the second total CBD/CBG/BAK molar concentration is greater than the first total CBD/CBG/BAK molar concentration.

27. The composition of claim 26, wherein the first total CBD/CBG/BAK molar concentration is in a range of 9 μM to 11 μM.

28. The composition of claim 26, wherein the second total CBD/CBGBAK molar concentration is greater than 11 μM.

29. The composition of claim 26, wherein the composition has a total CBD/CBG/BAK molar concentration of 10 μM.
